(19) United States
(12) Patent Application Publication
(10) Pub. No.: US 2004/0230315 A1
(43) Pub. Date: Nov. 18, 2004

(54) Articular surface implant

(76) Inventor: Steven W. Ek, Bolton, MA (US)

Correspondence Address: Grossman, Tucker, Perreault & Pfleger, PLLC, 55 South Commercial Street, Manchester, NH, 03101, US

(21) Appl. No.: 10/789,545

(22) Filed: Feb. 27, 2004

Related U.S. Application Data
(63) Continuation-in-part of application No. 10/373,463, filed on Feb. 24, 2003.
Application No. 10/373,463 is a continuation-in-part of application No. 10/162,533, filed on Jun. 4, 2002, now Pat. No. 6,679,917.
Application No. 10/162,533 is a continuation-in-part of application No. 10/024,077, filed on Dec. 17, 2001, now Pat. No. 6,610,067.
Application No. 10/024,077 is a continuation-in-part of application No. 09/846,657, filed on May 1, 2001, now Pat. No. 6,520,964.
(60) Provisional application No. 60/201,049, filed on May 1, 2000.

(51) Int. Cl. A61F 2/28

(52) U.S. Cl. 623/23.51; 623/901; 264/478

(57) ABSTRACT

An implant is provided for replacing a portion of an articular surface. The implant includes a body portion having a recess. An insert is disposed in the recess. The body portion and insert provide a load bearing surface of the implant, with the load bearing surface including regions of the insert and regions of the body portion. The insert may be formed from a lubricious and shock absorbing material, providing a low friction and shock absorbing region of the load bearing surface.

### FIELD

The present disclosure is generally directed at implants for replacing a portion of an articular surface, and more particularly, the present disclosure pertains to a composite implant for replacing a portion of an articular surface.

### BACKGROUND

Articular cartilage, found at the ends of articulating bone in the body, is typically composed of hyaline cartilage, which has many unique properties that allow it to function effectively as a smooth and lubricious load-bearing surface. However, when injured, hyaline cartilage cells are not typically replaced by new hyaline cartilage cells. Healing is dependent upon the occurrence of bleeding from the underlying bone and formation of scar or reparative cartilage called fibrocartilage. While similar, fibrocartilage does not possess the same unique aspects of native hyaline cartilage and tends to be far less durable.

Hyaline cartilage problems, particularly in knee and hip joints, are generally caused by disease such as occurs with rheumatoid arthritis or wear and tear (osteoarthritis). Hyaline cartilage problems may also be the result of an injury, either acute (sudden) or recurrent and chronic (ongoing). Such cartilage disease or deterioration can compromise the articular surface causing pain and further deterioration of joint function. As a result, various methods have been developed to treat and repair damaged or destroyed articular cartilage.

For smaller defects, traditional options for this type of problem include non-operative herapies (e.g., oral medication or medication by injection into the joint), or performing a surgical procedure called abrasion arthroplasty or abrasion chondralplasty. The principle behind this procedure is to attempt to stimulate natural healing. At the defect site, the bone surface is abraded, removing approximately 1 mm. or less using a high-speed rotary burr or shaving device. This creates an exposed subchondral bone bed that will bleed and will initiate a fibrocartilage healing response. Although this procedure has been widely used over the past-two decades and can provide good short term results, (1-3 years), the resulting fibrocartilage surface is seldom able to support long-term weight bearing, particularly in high-activity patients, and is prone to wear.

Another procedure, referred to as the “microfracture” technique, incorporates similar concepts of creating exposed subchondral bone. During the procedure, the cartilage layer of the chondral defect is removed. Several pathways or “microfractures” are created to the subchondral bleeding bone bed by impacting a metal pick or surgical awl at a minimum number of locations within the lesion. By establishing bleeding in the lesion and by creating a pathway to the subchondral bone, a fibrocartilage healing response is initiated, forming a replacement surface. Results for this technique are generally similar to abrasion chondralplasty.

Another known option to treat damaged articular cartilage is a cartilage transplant, referred to as a Mosaicplasty or osteoarticular transfer system (OATS) technique. This technique involves using a series of dowel cutting instruments to harvest a plug of articular cartilage and subchondral bone from a donor site, which can then be implanted into a core made into the defect site. By repeating this process, transferring a series of plugs, and by placing them in close proximity to one another, in mosaic-like fashion, a new grafted hyaline cartilage surface can be established. The result is a hyaline-like surface interposed with a fibrocartilage healing response between each graft.

Such an OATS procedure is technically difficult, as all grafts must be taken with the axis of the harvesting coring drill being kept perpendicular to the articular surface at the point of harvest. Also, all graft placement sites must be drilled with the axis of a similar coring tool being kept perpendicular to the articular surface at the point of implantation. Further, all grafts must be placed so that the articular surface portion of these cartilage and bone plugs is delivered to the implantation site and seated at the same level as the surrounding articular surface. If these plugs are not properly placed in relation to the surrounding articular surface, the procedure can have a very detrimental effect on the mating articular surface. If the plugs are placed too far below the level of the surrounding articular surface, no benefit from the procedure will be gained. Further, based on the requirement of perpendicularity on all harvesting and placement sites, the procedure requires many access and approach angles that typically require an open field surgical procedure. Finally, this procedure requires a lengthy post-operative non-weight bearing course.

Transplantation of previously harvested hyaline cartilage cells from the same patient has been utilized in recent years. After the cartilage is removed or harvested, it is cultured in the lab to obtain an increase in the number of cells. These cells are later injected back into the focal defect site and retained by sewing a patch of periosteal tissue over the top of the defect to contain the cells while they heal and mature. The disadvantages of this procedure are its enormous expense, technical complexity, and the need for an open knee surgery. Further, this technique is still considered somewhat experimental and long-term results are unknown. Some early studies have concluded that this approach offers no significant improvement in outcomes over traditional abrasion and microfracture techniques.

### BRIEF DESCRIPTION OF THE DRAWINGS

Features and advantages of the composite articular surface implant of the present invention are set forth by description of exemplary embodiments consistent with the present invention, which description should be considered in conjunction with the accompanying drawings, wherein:

FIG. 1 is a perspective view of a first exemplary composite implant consistent with the present disclosure;

FIG. 2 shows the exemplary composite implant of FIG. 1 in an exploded perspective view;

FIG. 3 is an exploded cross-sectional view of the exemplary composite implant of FIG. 1;

FIG. 4 is a cross-sectional view of the exemplary composite implant of FIG. 1;

FIG. 5 is a perspective view of a second exemplary composite implant consistent with the present disclosure;

FIG. 6 is an exploded perspective view of the exemplary composite implant illustrated in FIG. 5;

FIG. 7 illustrates the exemplary composite implant of FIG. 5 in side elevation;

FIG. 8 is a cross-sectional view of the exemplary composite implant of FIG. 5;

FIG. 9 is a perspective view of a third exemplary composite implant consistent with the present disclosure;

FIG. 10 shows the exemplary composite implant of FIG. 9 in an exploded perspective view;

FIG. 11 is a cross-sectional view of the exemplary composite implant of FIG. 9;

FIG. 12 is an exploded cross-sectional view of the exemplary composite implant of FIG. 9;

FIG. 13 is a perspective view of yet another exemplary composite implant consistent with the present disclosure;

FIG. 14 is an exploded perspective view of the exemplary composite implant of FIG. 13;

FIG. 15 shows the exemplary composite implant of FIG. 13 in an exploded cross-sectional view; and

FIG. 16 is a cross-sectional view of the exemplary composite implant shown in FIG. 13.

### DESCRIPTION

The present disclosure generally pertains to implants for replacing portions of an articular surface. Generally, a portion of an articular surface including a defect, or within a defect, may be excised creating an implant site. An articular surface implant may be installed in the implant site to resurface the excised portion of the articular surface. One suitable exemplary method is disclosed in U.S. Pat. No. 6,679,917, assigned to the assignee herein.

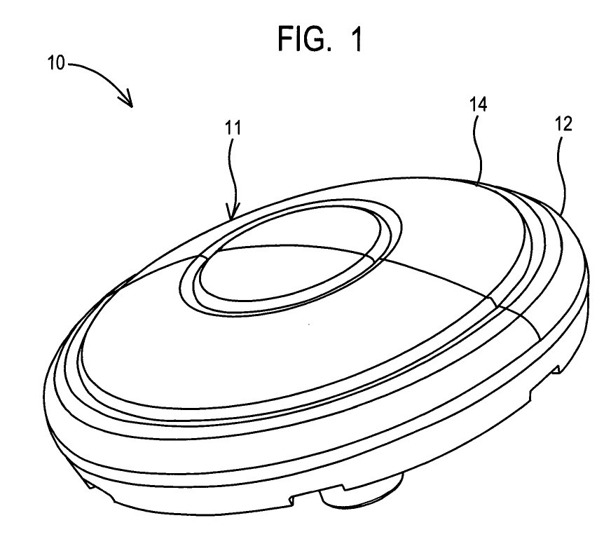

FIGS. 1 though 4 illustrate a first exemplary embodiment of a composite implant 10. The exemplary implant 10 may have a generally circular shape and provide an upper load bearing surface 11. The load bearing surface 11 of the implant 10 may provide a replacement articular surface. In the illustrated embodiment, the load bearing surface 11 of the exemplary implant 10 may have a generally arcuate contour or geometry. It will be appreciated, however, that the shape of the load bearing surface 11 may vary based on a specific application. The exemplary composite implant 10 may generally include an implant body 12 and an implant insert 14. The load bearing surface 11 of the implant may include at least a portion of the implant body 12 and at least a portion of the insert 14. In the illustrate embodiment, the load bearing surface 11 may include an annular region including the insert 14 with a central region and a marginal region including the implant body 12.

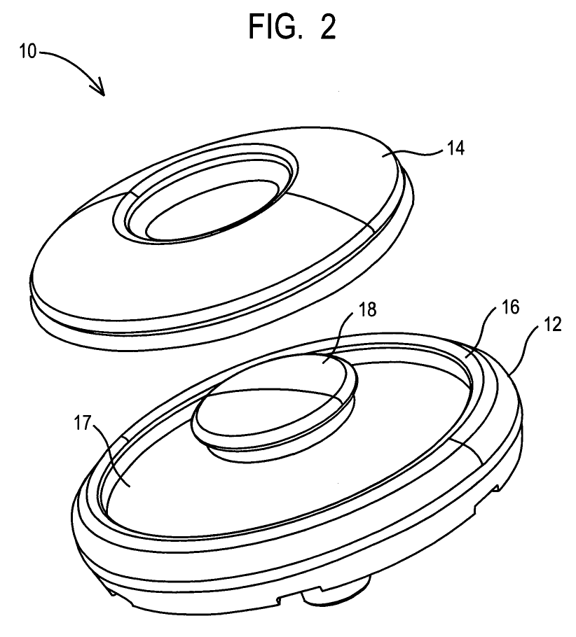

As best seen in FIG. 2, the insert 14 of the exemplary implant 10 may have an annular shape. The implant body 12 may include a rim 16 extending upwardly around the circumference of the implant 10, and may include an upwardly extending central member 18. A pocket or recess 17, adapted to at least partially receive the insert, may be defined between the rim 16 and the central member. According to one embodiment, the central member 18 may provide load absorption at the center of the implant 10.

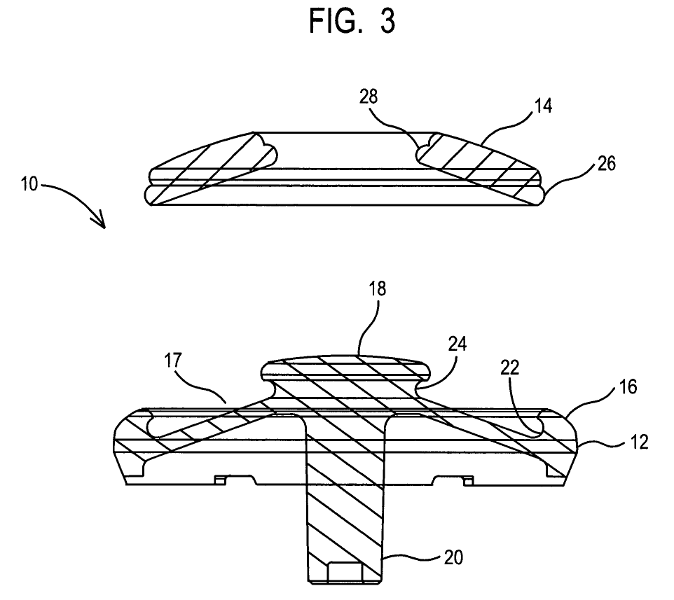

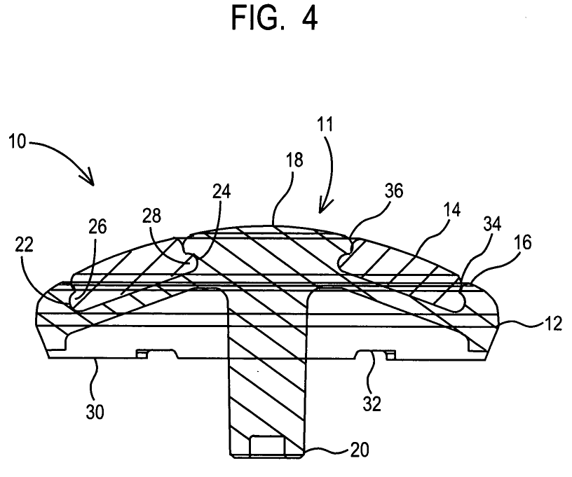

With reference to FIGS. 3 and 4, the implant body 12 may include an undercut or groove 22 around the inside of the rim 16. The insert 14 may include a cooperating bead 26 around the outside circumference of the insert. Similarly, the central member 18 of the implant body 12 may also include an undercut or groove 24 that is configured to receive a bead 28 around the inside diameter of the insert 14. As depicted in FIG. 4, the beads 26, 28 of the insert 14 may be received in the respective undercut grooves 22, 24 of the implant body 12, thereby mechanically locking the insert 14 and implant body 12 together.

The size and shaped of the cooperating undercuts 22, 24 and beads 26, 28 may be selected in consideration of the material of the insert 14 to provide a positive mechanical lock. For example, an insert 14 produced from a more rigid material may require a relatively shallow groove and bead to achieve a positive mechanical lock. By contrast, an insert 14 produced from a more flexible material may require a deeper groove and bead to securely connect the insert 14 to the implant body 12.

The edges of the insert 14 and/or the implant body 12 may be relieved along the interfaces 34, 36 between the insert 14 and implant body 12 on the load bearing surface 11. For example, the edges of the insert and implant body may be chamfered or rounded over on the load bearing surface 11 at the interfaces 34, 36. According to one aspect, relieving the edges may reduce the occurrence of a hard edge that may scrape an interacting articular surface. Additionally, relieving the edges may accommodate manufacturing tolerances. If an edge of the insert 14 is recessed or stands proud relative to the adjacent edge of the implant body 12, relieving the edge of the insert and/or the implant body may still allow smooth movement of an interacting articular surface across the interfaces 34, 36 on load bearing surface 11 of the implant 10. As illustrated, relieving the edges of the insert 14 and/or the implant body 12 may produce an indentation or witness on the load bearing surface 11 at the interfaces 34, 36 between the insert 14 and the implant body 12.

The implant body 12 may include a mounting feature 20, allowing the implant 10 to be secured to bone underneath the articular surface being replaced by the implant 10. According to the exemplary implant 10, the mounting feature 20 may include a tapered post extending from the bottom, or bone facing, surface of the implant body 12. The tapered post 20 may be received in a mating tapered recess, for example of a screw installed in the bone at an implant site. Various other mounting features may suitably be used to secure the implant 10 to either a fixturing element installed in the bone or to secure the implant to the bone itself.

The implant 10 may also include a ring 30 extending from a bone facing surface of the implant body 12. The ring 30 may be dimensioned to be at least partially received in an excised implant site. The ring 30 may include radial slots 32 spaced around the circumference of the ring 30. The slots 32 may aid anchoring the implant 10 to the bone at an implant site.

Consistent with the exemplary embodiment, the implant 10 may provide a load bearing surface including two or more materials. The implant 10 may replace at least a portion of an articular surface, wherein the replacement articular surface provided by the implant includes at least a portion of the load bearing surface. In one embodiment, the two or more materials may provide different properties, and allow the characteristics of the implant load bearing surface, and thereby the replacement articular surface, to be tailored to provide a more suitable replacement articular surface.

The composite implant 10 may provide an arrangement in which the insert 14 may be at least partially surrounded and supported by the implant body 12. With the implant body 12 at least partially surrounding and supporting the insert 14, the insert may be protected against mechanical overloading. The protection provided by the implant body in a composite implant configuration may provide an advantageous combination of properties, and/or extend the useful life of the implant relative to convention implants having a load bearing surface comprising a single material. It should be understood, however, that the insert 14 need not be completely surrounded or captured by the implant body 12.

Materials well known in the field of orthopedics may be used for the implant body 12. For example, stainless steel, titanium, cobalt-chromium alloys, etc. may be suitable for producing the implant body 12. The implant body may be manufactured using conventional processes, such as machining, casting, sintering, etc.

An insert consistent with the present disclosure may be formed from a material that is selected to provide specific characteristics. For example, the insert material may be selected to provide a low friction surface or to provide wear resistance. Additionally, the insert material may be selected to provide at least some degree of shock absorption or cushioning effect. The insert may include various biocompatible polymeric materials. Suitable materials may include various polymeric materials, for example, high density polyethylene, ultrahigh molecular weight polyethylene, polyurethane, polyhydroxy-ethyl methacrylate gel, silicone, polyvinyl alcohol gel, etc. Ceramic materials, such as alumina or zironia based materials, may also be used, e.g., to provide an inherent lubrication or low friction surface. Additionally, the insert may include materials that release or produce therapeutic or lubricating products and may even include biological materials. Those having skill in the art will numerous other materials that may be used to produce the inserts according to the present disclosure. In an exemplary embodiment, the insert may be formed from a hydrogel material. An exemplary suitable hydrogel material, may include polyvinyl alcohol hydrogel.

According to one embodiment, the insert 14 may be a molded component produced using convention molding operations, such as compression molding, injection molding, and casting. The insert 14 may also be cut from a sheet or block of the insert material, e.g., using a computer controlled cutting operation, die stamping, etc. The insert 14 may then be pressed into the recess or pocket 17 formed in the implant body 12, until the beads 26, 28 engage with the undercuts 22, 24. When the beads 26, 28 are engaged with the undercuts 22, 24 the insert may remain installed in the implant 10 for the useful life of the implant.

According to another embodiment, the insert 14 may be molded or cast directly into the recess or pocket 17 of the implant body 12. For example, if the insert 14 is formed from an injection-moldable material, the implant body 12 may be supported in a molding arrangement such that a mold portion and the implant body 12 define molding cavity. The mold portion may define the load bearing surface of the insert, and the recess or pocket 17 of the implant body 12 defines the remainder of the insert 14. The insert material may be injected directly into the molding cavity. After the insert material has at least partially solidified, the implant 10 including an in situ molded insert 14 may be removed from the molding arrangement. This process may be generally similar to a conventional insert molding process. Similar manufacturing methods may be achieved using casting processes, compression molding processes, etc.

The illustrated implant 10 includes a load bearing surface 11 that is largely formed by the annular insert 14. Consistent with related embodiments, the annular insert may provide a smaller proportion of the load bearing surface. Accordingly, the implant body may include a larger central member and/or a wider rim. In some embodiments, the annular insert may not be centered on the load bearing surface, but rather eccentrically disposed thereon.

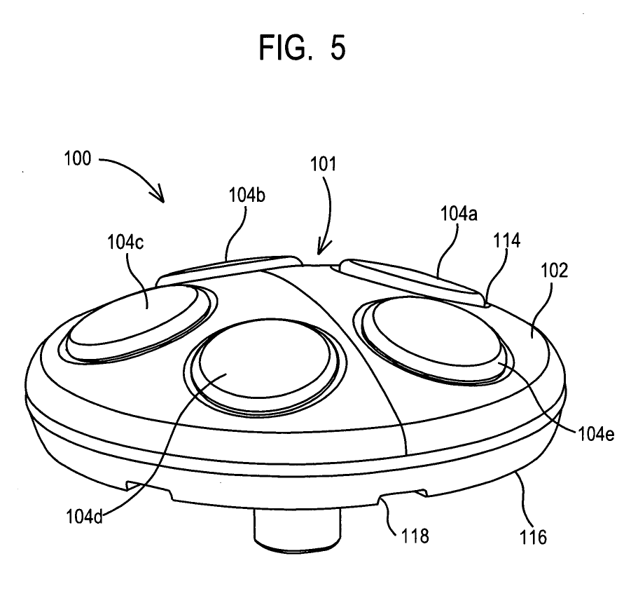

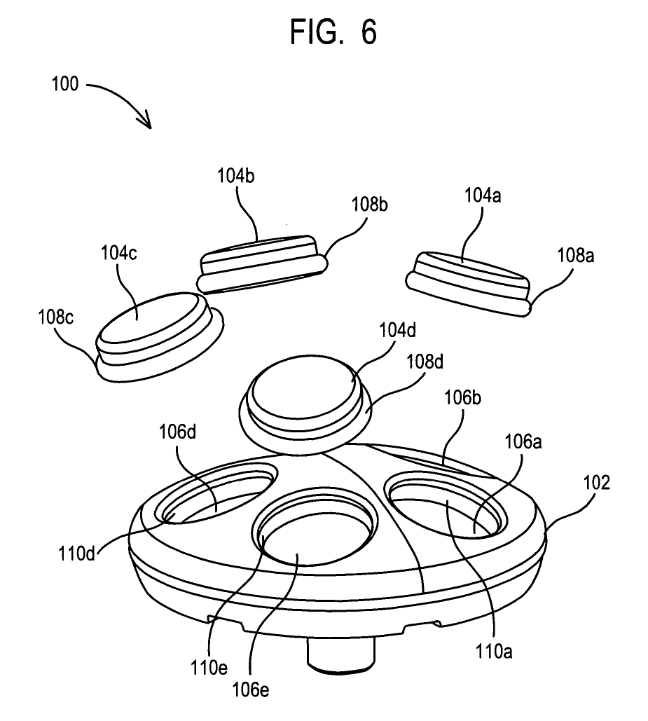

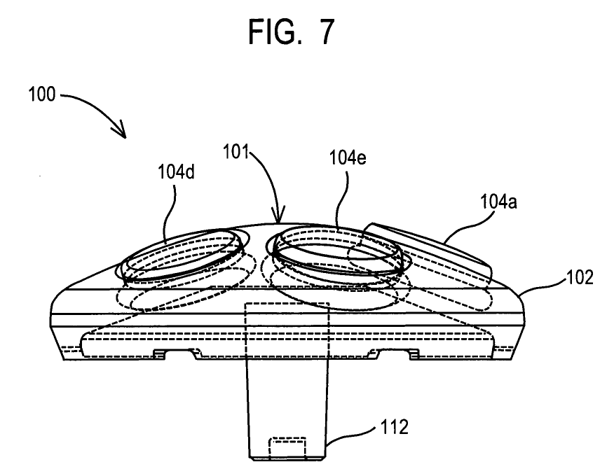

Turning to FIGS. 5 through 8 another exemplary composite implant 100 is shown. The implant may include an implant body 102 including a plurality of inserts 104a-e. Accordingly, the implant 100 includes a load bearing surface 101 having a plurality of inserts 104a-e providing discrete regions or pads of the insert material on the load bearing surface.

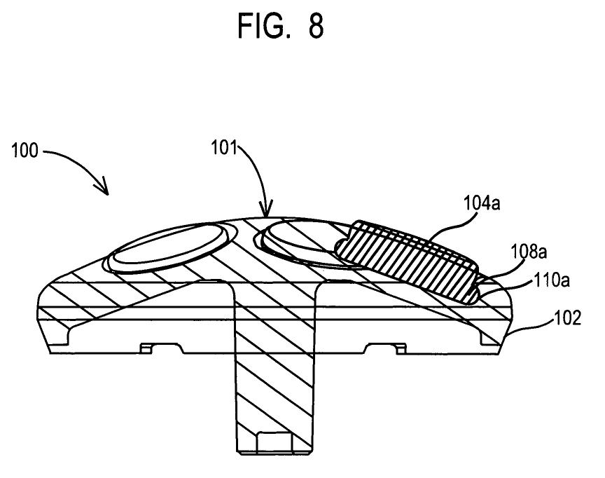

The implant body 102 may include pockets or recesses 106a-e in the load bearing surface 101 for receiving the inserts 104a-e. The pockets 106a-e may each contain an undercut or groove 110a-e in the sidewall of the pocket 106a-e. The inserts 104a-e may each include a bead 108a-e or protrusion. As shown in FIG. 8, when an insert 104a is disposed in a pocket 106a, the bead 108 may engage the groove 110a-e. The inserts 1-4a-e may therefore be retained in the pockets 106a-e and may be supported by the implant body 102.

The implant 100 may include many aspects similar to the previously described embodiment. The inserts 104a-e may be separately cut or molded and then pressed into the pockets or recesses 106a-e of the implant body 102. The edges of the inserts 104a-e and/or pockets 106a-e may be relieved on load bearing surface 101 at the interface between the insert 104 and implant body 102. Additionally, the implant body 102 may include a mounting feature 112, allowing the implant 100 to be secured to bone at an implant site. The implant body 102 may include a downwardly extending rim 116, which may include radial slots 118, to facilitate anchoring the implant 100 to the bone at the implant site.

In the illustrated exemplary implant 100 the inserts 104a-e are shown generally evenly spaced around the load bearing surface 101 of the implant 100. According to another embodiment, an implant may be provided in which the inserts are disposed having varying radial and/or angular spacing. Additionally, the illustrated implant 100 includes inserts 104a-e that are shown to be generally the same size. However in some applications it may be advantageous to provide an implant including a plurality of inserts having different sizes. Such embodiments are contemplated by the present disclosure.

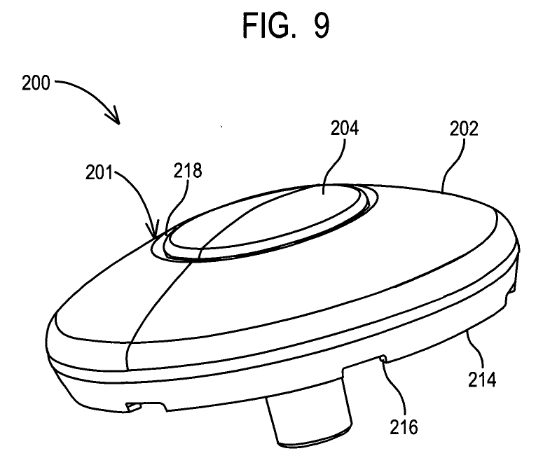

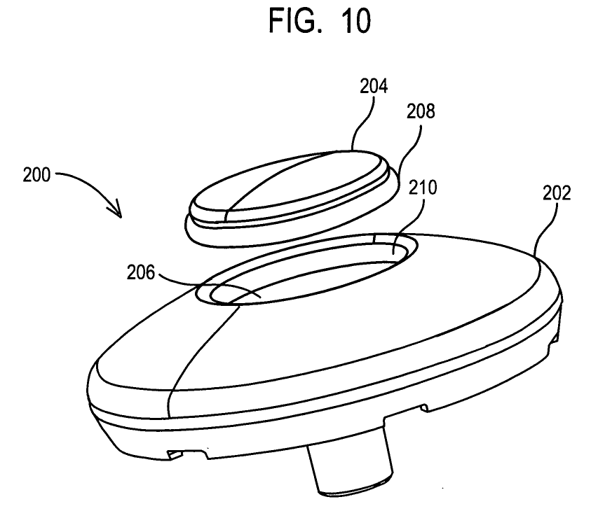

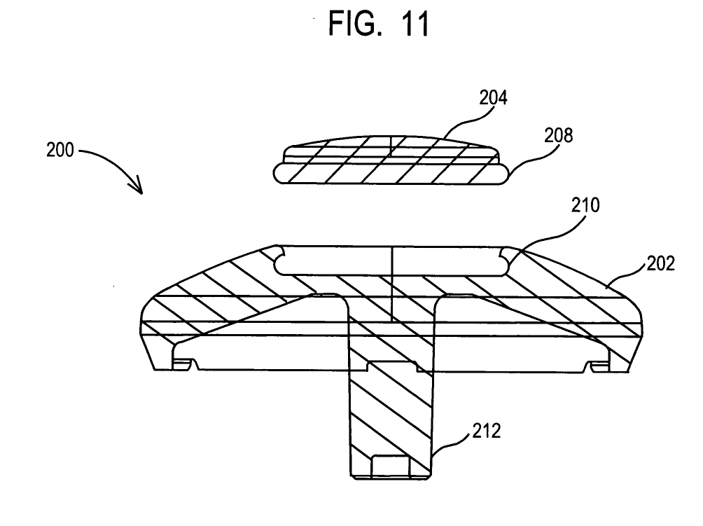

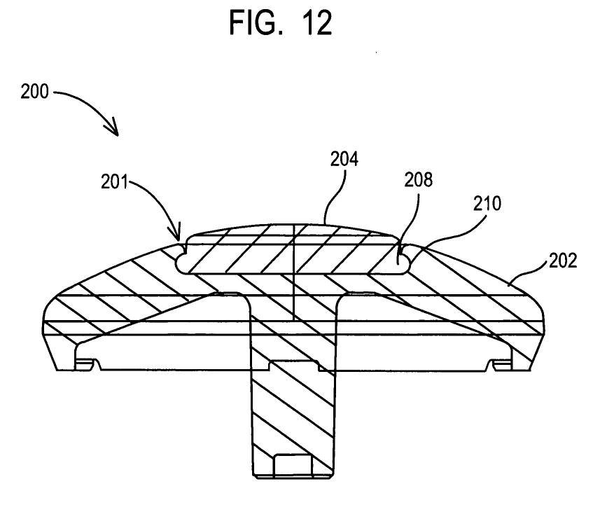

Referring to FIGS. 9 through 12, another embodiment of a composite implant 200 is illustrated. The composite implant 200 may include an implant body 202 having an insert 204 disposed generally in the center of the load bearing surface 201 of the implant 202. The insert 204 may be received in a pocket or recess 206 in the implant body 202. Similar to the previous embodiment, the insert 204 may include a bead or protrusion 208 extending generally around the circumference of the insert 204. The recess 206 in the implant body 202 may include a groove or undercut 210 configured to receive the bead 208 on the insert 204 when the insert 204 is disposed in the recess 206.

The implant 200 may also include a mounting feature 212 for locating and/or securing the implant in desired location. A downwardly extending rim 214, which may include radial slots 216, may be provided to facilitate anchoring the implant 200 to an implant site. Additionally, on the load bearing surface 201, the insert 204 and/or the implant body 202 may include relieved edges at the interface 218 of the insert 204 and implant body 202.

The exemplary implant illustrated in FIGS. 9 though 12 shows the insert located in the center of the load bearing surface. It should be appreciated, however, that this embodiment is susceptible to variation. The insert may be provided at other locations on the load bearing surface to tailor the implant to specific applications.

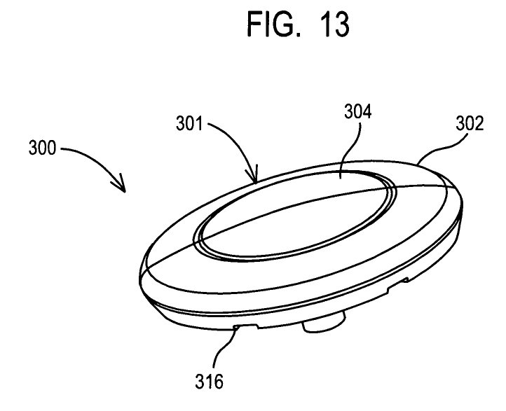

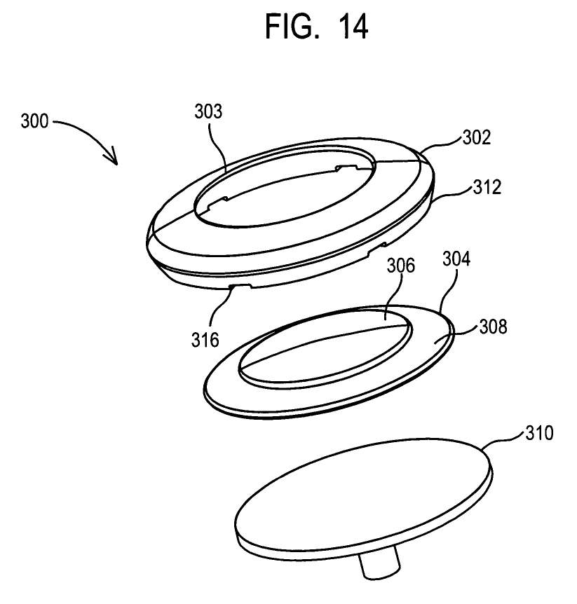

Turning to FIGS. 13-16 another embodiment of a composite implant 300 is depicted. The implant 300 may include an implant body which may include a top member 302 and a support member 310. An insert 304 may be disposed at least partially between the top member 302 and the support member 310. The top member 302 may include at least one opening 303 exposing at least a portion of the insert 304. Accordingly, the load bearing surface 301 may be provided including both the top member 302 and the insert.

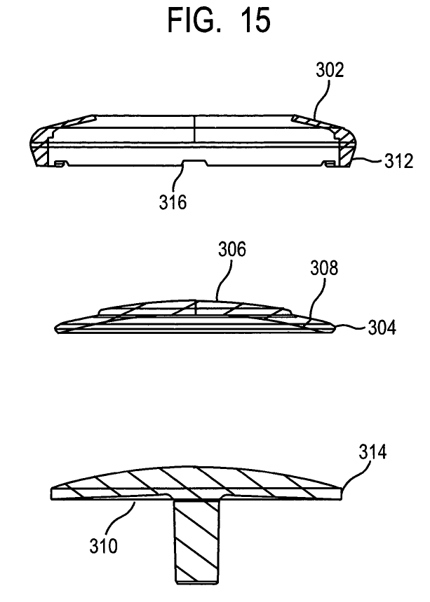

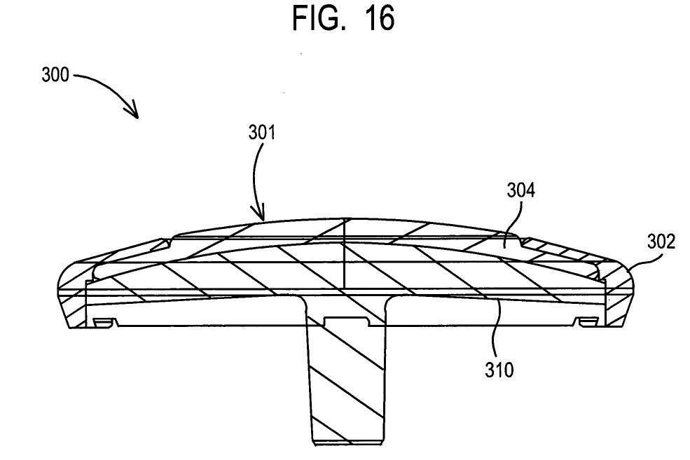

Referring to the cross-sectional views of the implant 300 shown in FIGS. 15 and 16, the top member 302 may include a downwardly extending rim 312. The rim 312 may be sized to at least partially receive a backing member 314 of the top member 310. According to one embodiment, when the top member 302 is assembled to the support member 310, the rim 312 of the top member 302 may extend below the backing member 314, thereby providing the implant body with a rearwardly extending rim that may engage bone beneath the articular surface receiving the implant 300. The rim 312 may include a plurality of radial slots 316 that may aid anchoring the implant to the bone beneath the articular surface.

The insert 304 consistent with this embodiment may include a flange 308 extending round at least a portion of the insert 304. When assembled the flange 308 may be disposed between the implant top member 302 and the implant support member 310, thereby at least partially capturing the insert 304. A raised contact surface 306 of the insert 304 may be received in the opening 303 of the top member 302, thereby providing a generally continuous load bearing surface 301 including the top member 302 and the insert 304.

As with the preceding embodiments, the insert 304 may be molded, cast, or cut from appropriate stock. The insert 304 may be disposed between the top member 302 and the support member 310. The top member 302 and the support member 310 may be assembled, e.g., using a press-fit, welding, adhesive bonding, etc., to capture at least a portion of the insert 304 therebetween. The flange 308 of the insert 304 disposed between the top member 302 and the support member 310 may retain the insert 304 in the implant 300. The contact surface 306 of the insert 304 may be circumferentially supported by the opening 303 of the top member 302, and may also be supported from behind by the backing portion 314 of the support member. The top member 302 may be assembled to the support member 310 at least partially compressing the flange 308. Compressing the flange 308 between the top member 302 and the support member 310 may also assist retaining the insert 304 in the implant 300.

Also as discussed with reference to preceding embodiments, the insert 304 may be cast or molded in situ between the top member 302 and the support member 310. The top member 302 may be assembled to the support member 310, providing a recess therebetween for the insert. The assembled top member 302 and support member 310 may be supported in a molding arrangement including a mold portion defining the load bearing surface of the insert. The assembled top member 302 and support member 310 may define the remainder of the insert. Accordingly, a molding cavity for the insert may be provided by the combination of the mold portion, the top member 302 and the support member 310. Insert material may be introduced into the molding cavity, thereby forming the insert in situ.

The embodiment illustrated in FIGS. 13 through 16 may be especially advantageous for providing thin implants. A thin implant may provide a more shallow recess for retaining an insert. When the thickness of the insert relative to diameter or area of the insert is decreased, the insert may become too flexible to be retained by a bead disposed in an undercut. That is, in the insert may tend to deform causing the insert to release from the implant body. Capturing the insert in a manner according to the embodiment of FIGS. 13 through 16 may provide enhanced retention of the insert.

Further embodiments may be provided employing an insert having a flange retained by an implant body having a top member and a support member. For example, an implant may be provided having an annular insert exposed on the load bearing surface, similar to the embodiment shown in FIG. 1. The insert may include an annular member having a flange extending into the inside diameter of the insert and a flange extending from the outside diameter of the insert. Another exemplary embodiment may provide a load bearing surface including more than one region of the insert, similar to the embodiment illustrated in FIG. 5. Such an embodiment may be provided using multiple flanged inserts exposed or received through multiple openings in the implant body top member. Alternatively, the implant may be provided including one insert having multiple contact surfaces that may be exposed or received through multiple openings in the implant body top member.

The various exemplary embodiments herein describe articular surface implants having a load bearing surface including an implant body and an insert. In the several embodiments the insert may be provided having a variety of configurations and in a variety of locations on the load bearing surface. It should be understood that the configuration of the insert and the location of the insert may be varied to achieve different objectives and to suit different applications.

In one example, an insert of an implant may be employed to provide shock absorption characteristics. An insert having improved shock absorption characteristics, relative to the implant body, may be provided at a location on the implant the experiences the shock or impact loading. The insert may at least partially absorb any shock to the implant. The implant body may support the insert to prevent mechanical overloading of the insert. For example, the implant body may limit or control the amount of deformation experienced by the insert. The implant body may, therefore decrease the deterioration of the insert.

According to another example, the insert may be employed to provide a lubricious contact surface. The implant body may be provided in a location experiencing the greatest shock or impact loading, thereby providing greater durability. The insert having low friction or lubricating characteristics may be provided in adjacent regions of the implant to provide a low friction with interaction articular surfaces.

The exemplary embodiments described and illustrated are generally directed at implants having a generally circular shape and presenting a convex load bearing surface. These aspects are not critical within the present disclosure. The shape of the implant and the geometry of the load bearing surface may be varied to suit different applications. For example, an elongated implant may be provided to replace a portion of the articular surface in which the region of the articular surface being replaced is longer in one plane than the width of the articular surface in a transverse plane. In various applications, it may also be desirable to provide non-symmetrical implants.

An implant according to the present disclosure may be provided having a load bearing surface that may approximate the geometry or curvature of the articular surface being replaced by the implant. In one embodiment the geometry of the load bearing surface may be based on the actual articular surface being replaced. For example, mapping techniques known in the art may be used to measure the geometry of the region of the actual articular surface being replaced. An implant may then be constructed or selected from a set of implants having predetermined geometries. Alternatively, an implant for a specific application may be fabricated or selected from a set of standard sized/shaped implants to provide a general approximation of the articular surface being replaced. Selection or fabrication of an implant may rely on various degrees of quantitative reference to the articular surface being replaced, including no quantitative reference to the articular surface.

Different articular surfaces, and even different regions of an articular surface, may be susceptible to replacement by implants having load bearing surface of various geometries. In some application a convex load bearing surface may be suitable. However, in other applications a planar, concave, or compound curve load bearing surface may provide a more suitable implant geometry. Additionally, while the exemplary embodiments generally show the load bearing surface oriented perpendicular to the mounting feature, it may be desirable to provide the load bearing surface having an angular orientation relative to the mounting feature.

Various exemplary embodiments of implants according to the present disclosure employ inserts in a variety of configurations and placements on the load bearing surface. Consistent with one aspect, inserts may be provided having a standard or generic size and shape, or an array of standard sizes and shapes. Sets of implant bodies having different configurations and sets of inserts within the array of standard sized and shapes may be stocked. Individual implants may be custom assembled for specific applications from the stock of implant bodies and inserts. Accordingly, a variety of different implants may be provided without the need for producing inserts specially configured for each implant body. This design approach may allow the manufacturing burden to be reduced.

According to another aspect, an implant may be provided having a replaceable insert portion. Time, use, injury, etc. may cause even relatively long wearing materials, such as ultra-high molecular weight polyethylene or ceramic, to wear or break down, or otherwise assume a condition that provides less than desired performance of the implant. If the implant performance deteriorates below a desired level the insert may be configured to be replaced. During a surgical procedure, an insert may be fully or partially extracted from the implant and a new insert may be installed into the implant. For example, in the case of an implant having an insert pressed into a recess or pocket, after the previously installed insert has been removed from the implant a new insert may be pressed into the implant. In addition to replacing worn, damaged, etc. inserts, an insert may also be replaced in order to change the material or a characteristic of the insert. For example, and insert originally employed to provide a lubricious region of the implant may be replaced with an insert providing a cushioning or impact absorbing characteristic. It should also be noted that, in the case of an implant including more than one insert, it is not necessary to replace all of the inserts of an implant.

In view of the foregoing, it should be understood that the exemplary embodiments described herein are susceptible to modification and variation without materially departing from the present invention. Accordingly, the invention herein should not be limited by the described embodiments, but only by the claims appended hereto.

What is claimed is:

1. An implant comprising:
a load bearing surface, said surface comprising a body portion and an insert.

2. The implant according to claim 1, wherein body portion comprising a recess and said insert is at least partially disposed in said recess.

3. The implant according to claim 2, wherein said recess comprises an undercut and said insert comprises a protrusion at least partially received in said undercut.

4. The implant according to claim 1, further comprising a mounting feature.

5. The implant according to claim 1, wherein said load bearing surface comprises said body portion and a plurality of inserts.

6. The implant according to claim 1, wherein said body portion comprises a metallic material.

7. The implant according to claim 1, wherein said insert comprises a polymeric material.

8. The implant according to claim 7, wherein said insert comprises a hydrogel material.

9. The implant according to claim 1, said implant providing a replacement articular surface comprising at least a portion of said load bearing surface.

10. The implant according to claim 9 wherein said load bearing surface provides said replacement articular.

11. An implant comprising:
an implant body portion comprising at least one recess; and
an insert disposed in said recess;
said implant comprising a load bearing surface, said load bearing surface comprising a portion of said body portion and a portion of said insert.

12. The implant according to claim 11, wherein said body portion comprises a metallic body.

13. The implant according to claim 11, wherein said insert comprises a polymeric material.

14. The implant according to claim 13, wherein said insert comprises a hydrogel material.

15. The implant according to claim 14, wherein said insert comprises polyvinyl alcohol hydrogel.

16. The implant according to claim 11, wherein said recess comprises an undercut and said insert comprises a protrusion adapted to be at least partially received in said undercut.

17. The implant according to claim 11, wherein said recess comprises an annular recess and said insert comprises an annular insert.

18. The implant according to claim 11, wherein said body portion comprises a first member comprising an opening and a second member, said recess at least partially defined between said first and second members, said insert disposed at least partially between said first and second members.

19. The implant according to claim 11, said body portion comprising a plurality of recesses, and said implant comprising a plurality of inserts respectively disposed in said plurality of recesses.

20. The implant according to claim 11, wherein said body portion comprises a mounting feature.

21. The implant according to claim 11, wherein said body portion comprises a rim extending away from said load bearing surface, said rim comprising at least one radial slot.

22. A method of producing a composite implant comprising:
providing an implant body comprising a recess, said recess including an undercut;
providing an insert adapted to be at least partially received in said recess, said insert comprising a protrusion adapted to be received in said undercut; and
installing said insert in said recess.

23. The method according to claim 22, wherein providing said insert comprises molding said insert.

24. The method according to 23, wherein molding said insert comprises injection molding.

25. The method according to claim 22, wherein providing said insert comprises cutting said insert from a body of stock.

26. The method according to claim 22, wherein installing said insert in said recess comprises positioning said protrusion at least partially in said undercut.

27. The method according to claim 22, wherein said implant body comprises a first member and a second member, said first and second members defining a recess therebetween.

28. The method according to claim 27, wherein installing said insert in said recess comprises disposing said insert between said first and second members.

29. A method for producing a composite implant comprising:
providing an implant body comprising a recess, said recess including an undercut;
introducing a polymeric material into said recess; and
at least partially solidifying said polymeric material.

30. The method according to claim 29, wherein introducing a fluid polymeric material comprises injecting a molten polymeric material into said recess.

31. The method according to claim 29, further comprising providing a mold portion adjacent said recess, thereby defining a molding cavity defined by said recess and said mold portion.

32. The method according to claim 29, wherein introducing said polymeric material comprises introducing a powdered polymeric material, the method further comprising heating said polymeric material.
